(19) United States
(12) Patent Application Publication
(10) Pub. No.: US 2008/0202195 A1
(43) Pub. Date: Aug. 28, 2008

(54) GOLD NANOPARTICLE-BASED pH SENSOR IN HIGHLY ALKALINE REGION BY SURFACE-ENHANCED RAMAN SCATTERING STUDY

(75) Inventors: Sang Woo JOO, Seoul (KR); Jong Kuk LIM, Seoul (KR)

Correspondence Address: CANTOR COLBURN, LLP, 20 Church Street, 22nd Floor, Hartford, CT, 06103, US

(73) Assignees: SAMSUNG ELECTRONICS CO., LTD., Suwon-si (KR); SEOUL NATIONAL UNIVERSITY INDUSTRY FOUNDATION, Seoul (KR)

(21) Appl. No.: 11/835,279

(22) Filed: Aug. 7, 2007

(30) Foreign Application Priority Data
Aug. 18, 2006 (KR) 10-2006-0078246

(51) Int. Cl. G01N 33/00

(52) U.S. Cl. 73/1.02

(57) ABSTRACT

Disclosed are a pH sensor for use in a highly alkaline region of pH >11 comprising citrate-reduced gold nanoparticles and a method for calibrating pH of a solution in highly alkaline regions, based on variation in surface-enhanced Raman scattering spectra (SERS).

This application claims priority to Korean Patent Application No. 2006-78246, filed on Aug. 18, 2006 and all the benefits accruing therefrom under 35 U.S.C. § 119, the content of which is incorporated herein by reference in its entirety.

### BACKGROUND OF THE INVENTION

1. Field of the Invention

Embodiments of the invention relate to a gold nanoparticle-based pH sensor, and in particular to a gold nanoparticle-based pH sensor in a highly alkaline region based on variation in surface-enhanced Raman scattering spectra. Furthermore, example embodiments relate to a method for calibrating pH of a solution in highly alkaline regions, based on variation in surface-enhanced Raman scattering spectra.

2. Description of the Related Art

Gold nanoparticles have attracted much attention for several decades owing to stability, uniformity and optical properties⁽¹⁾. The optical properties of gold nanoparticle aggregates have been widely investigated by surface plasmon resonance (SPR) and transmission electron microscopy (TEM)⁽²⁾. The surface plasmon resonance (SPR) has been used to monitor the surface-binding interaction between a calorimetric sensor and an analyte⁽³⁾. The extent of aggregation for a molecular assembly of aliphatic thiol on gold nanoparticles was estimated from measurement of integrated absorbance at 600 to 800 nm⁽⁴⁾.

Since surface-enhanced Raman scattering (hereinafter, referred to simply as “SERS”) was considered as a highly sensitive spectroscopic technique for interface studies, it has been widely employed as a chemical sensor in the field of analytical chemistry⁽⁵⁻⁷⁾. SERS provides chemically characteristic information based on the specific vibration mode of a given target adsorbate. In recent years, in order to characterize gold nanoparticle aggregates, various experimental techniques e.g. SERS, quasi-elastic light scattering (QLES) and zeta-potential measurement have been studied⁽⁸⁾.

Self-assembled monolayers (hereinafter, referred to simply as “SAMs”) were considered important in nanoscience and nanotechnology owing to applicability to molecular scale electronics and biocompatibility. A pKa value on the interface of SAMs has been obtained by measurement of capacitance⁽⁹⁾, second harmonic generation⁽¹⁰⁾ and chemical force microscopy⁽¹¹⁾. The measurement of pH and pKa values has been conducted using SERS titration^{(12, 13)}. It was reported that modes of the pyridine ring in 4-mercaptopyridine are varied at weakly acidic regions of pH 3 to 6^{(14, 15)}. The SERS spectra of salicylic acid, pyridine and 2-naphthalenethiol were found to be closely connected with pH and zeta-potential⁽¹⁶⁾. An attempt to form organic SAMs containing no sulfur atom as an anchoring group on metal surfaces was hardly made to date⁽¹⁷⁾. There were reported several researches associated with metals or metal oxides on which amine-terminal⁽¹⁸⁾ and carboxylic acid-terminal⁽¹⁹⁾ SAMs are formed.

Anchoring of an aromatic ring via an alkynyl group has an advantage in that it provides a π-conjugation linkage to the metal surface⁽²⁰⁻²²⁾. The SERS of diethynylbenzene on gold and silver surfaces has been reported⁽²³⁻²⁵⁾. Multiple bands observed in v(C≡C) stretching regions was considered to be ascribed to adsorption on other crystals or presence of other complexes. However, reliable reasons for splitting are not clearly known to date. According to a recent report⁽²⁵⁾ by the inventors, it was found that variation of the multiple bands in C≡C stretching regions of SERS spectra is caused by addition of other ions to a sol medium.

Highly alkaline sensors are considerably valuable to the fields of environment⁽²⁶⁾ or biochemistry⁽²⁷⁾. To the best of our knowledge, there is no specific study that demonstrates usefulness of gold nanoparticles as a pH sensor in highly alkaline regions (i.e. pH >11). In particular, there is no report associated with pH calibration in highly alkaline regions using SERS titration based on v(C≡C) stretching bands.

### BRIEF SUMMARY OF THE INVENTION

The inventors confirmed the fact that gold nanoparticles exhibit a distinct color change in highly alkaline regions. This fact means that gold nanoparticles can be used as a pH sensor in highly alkaline regions. Therefore, example embodiments provide use of citrate-reduced gold nanoparticles as a pH sensor in a highly alkaline region (i.e. pH >11).

Furthermore, it was confirmed from surface-enhanced Raman scattering (SERS) of the gold nanoparticles, on which self-assembled monolayers composed of compounds having acetylene as an anchoring group and a pyridine ring are formed, that the multiple peaks in v(C≡C) stretching bands vary significantly according to pH variation in highly alkaline regions. This indicates that pH calibration of a solution can be carried out by measuring v(C≡C) stretching band intensities. Accordingly, example embodiments provide use of gold nanoparticles, on which self-assembled monolayers composed of compounds having a pyridine ring and acetylene as an anchoring group are formed, as a pH sensor in a highly alkaline region (i.e. pH >11).

Example embodiments provide pH calibration employing the gold nanoparticles.

Example embodiments also provide a pH sensor for use in a highly alkaline region of pH >11 comprising citrate-reduced gold nanoparticles.

Each citrate-reduced gold nanoparticle may have a surface on which a self-assembled monolayer composed of a compound having a pyridine ring and an acetylene group as an anchoring group is formed.

Example embodiments provide a method for calibrating pH of a solution in a highly alkaline region of pH >11, the method comprising the steps of: adding citrate-reduced gold nanoparticles to a target sample; obtaining surface-enhanced Raman scattering spectra from the sample; and calibrating pH of the sample via measurement of v(C≡C) stretching band intensity from the spectra, wherein each citrate-reduced gold nanoparticle has a surface on which a self-assembled monolayer composed of a compound having a pyridine ring and an acetylene group as an anchoring group is formed.

### BRIEF DESCRIPTION OF THE DRAWINGS

The above and other features and advantages of the embodiments will be more clearly understood from the following detailed description taken in conjunction with the accompanying drawings, in which:

FIGS. 1(a) and 1(b) are absorption spectra and a titration calibration curve of indigo carmine according to pH variation at about 610 nm, respectively;

FIGS. 2(a) to 2(c) are images showing color variation of gold nanoparticles in each region of pH <11, 11<pH<13 and pH >13.0, respectively, FIG. 2(d) a graph showing UV-vis spectra of gold nanoparticles in the regions, and FIG. 2(e) is a graph showing pH titration calibration curve corresponding to integrated values of the absorbance ranging from 600 to 800 nm;

FIG. 3(a) are absorption spectra i) before addition of 4-EP at pH 6.5, ii) after addition of 4-EP at pH 6.5, iii) after addition of 4-EP at pH 13.8, respectively, and FIGS. 3(b) to 3(d) are images of a sample under the following conditions: before addition of 4-EP at pH 6.5; after addition of 4-EP at pH 6.5; and after addition of 4-EP at pH 13.8, respectively;

FIG. 4 is a graph showing SERS spectra of 4-EP on gold nanoparticle surfaces according to pH variation, more specifically, at the following conditions: (a) pH 0.8, (b) pH 1.8, (c) pH 4.4, (d) pH 6.1, (e) pH 13.1, (f) pH 13.8, and (g) 4-EP in a liquid phase;

FIG. 5 is an enlarged view of v(C≡C) stretching SERS spectra of 4-EP on gold nanoparticle surfaces at respective pH: (a) pH 6.7, (b) pH 7.2, (c) pH 11.7, (d) pH 12.7, (e) pH 13.0, (f) pH 13.1, (g) pH 13.2, (h) pH 13.3, (i) pH 13.6, (j) pH 13.7 and (k) pH 13.8; and

FIGS. 6(a) and 6(b) are graphs showing a pH calibration curve with respect to a peak intensity ratio between two ν₈ₐ bands at 1,590 and 1,620 cm⁻¹ and between two C≡C bands at 2,080 and 2,010 cm⁻¹, respectively.

### DETAILED DESCRIPTION OF THE INVENTION

Hereinafter, embodiments will be explained in more detail with reference to the accompanying drawings.

It will be understood that when an element is referred to as being “on” another element, or “between” or “interposed between” other elements, it can be directly in contact with the other element, or intervening elements may be present therebetween. In contrast, when an element is referred to as being “disposed on” or “formed on” another element, the elements are understood to be in at least partial contact with each other, unless otherwise specified.

The terminology used herein is for the purpose of describing particular embodiments only and is not intended to be limiting of the invention. As used herein, the singular forms “a” “an” and “the” are intended to include the plural forms as well, unless the context clearly indicates otherwise. The use of the terms “first”, “second”, and the like do not imply any particular order but are included to identify individual elements. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In an attempt to develop a method for monitoring highly alkaline conditions (i.e. pH >11), the inventors investigated adsorption behaviors of a pyridine compound having acetylene as an anchoring group on gold nanocrystal surfaces.

Illustrated exemplary embodiments disclose a pH sensor for use in a highly alkaline region of pH >11 comprising citrate-reduced gold nanoparticles. The citrate-reduced gold nanoparticles exhibit a distinct color change in a highly alkaline region. The pH sensor according to an exemplary embodiment of the present invention, enables pH calibration in highly alkaline regions as well as more precise pH measurement, as compared to conventional indicators.

The gold nanoparticles have a surface on which a self-assembled monolayer composed of a compound having a pyridine ring and an acetylene group as an anchoring group is formed. Examples of the compound that can be used in the invention include, but are not limited to 4-ethynylpyridine having the structure shown in the following formula (I).

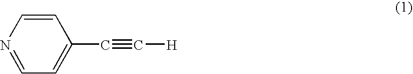

Another exemplary embodiment discloses a method for calibrating pH of a solution in a highly alkaline region of pH >11. In particular, the calibration of pH of a solution in a highly alkaline region according to an embodiment includes adding citrate-reduced gold nanoparticles to a target sample; obtaining surface-enhanced Raman scattering spectra from the sample; and calibrating pH of the sample via measurement of v(C≡C) stretching band intensity from the spectra. In this embodiment, each citrate-reduced gold nanoparticle has a surface on which a self-assembled monolayer composed of a compound having a pyridine ring and an acetylene group as an anchoring group is formed.

Examples of the compound that can be used in the invention include, but are not limited to 4-ethynylpyridine and the highly alkaline region ranges from pH 11.5 to 14.

In the present method, wherein the measurement of v(C≡C) stretching band intensity is carried out by calculating an intensity ratio between two characteristic peaks.

Exemplary embodiments are next described in more detail. However, these examples are provided for the purpose of illustration only and are not to be construed as limiting the scope of the invention.

### EXAMPLES

Citrate-reduced gold nanoparticles are synthesized according to the method disclosed in the reference document⁽²⁸⁾.

First, 133.5 mg of KAuCl₄ was dissolved in 250 mL of water, followed by boiling. A 1% sodium citrate solution (25 mL) was added to the KAuCl₄ solution with vigorous stirring. The solution was continuously boiled for about 20 min to obtain a colloidal solution of gold nanoparticles. With the exclusion of evaporated water, the concentration of gold (Au) particles was about 1.28×10⁻³ M and the concentration of gold nanoparticles was about 9.1×10⁻⁹ M⁽²⁹⁾. Unless otherwise specified, all chemicals are reagent grade and aqueous solutions are prepared from tertiary distilled water having a resistance of 18.0 MΩcm or more. To compare gold nanocrystal aggregates induced by SAMs, SAMs were formed on the surface of the gold nanoparticles by using 4-ethynylpyridine (4-EP) having the structure shown in the following formula (1), which is a pyridine compound having acetylene as an anchoring group.

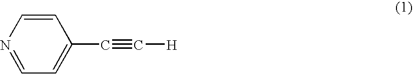

Characterization of Gold Nanocrystal Aggregates

A drop of the colloidal gold nanocrystal solution was placed on carbon-coated copper grids of a transmission electron microscope (Tecnai F20 Philips® or JEM-2000EXII®) and TEM images were then obtained. As a result of statistical analysis, the diameter of the gold nanocrystal was 15 nm or less. The UV-vis absorption spectra of the colloidal solution were obtained with a Shimazu UV-3101PC spectroscope. The maximum wavelength (λₘₐₓ) of the spectra was about 520 nm. The full width half maximum (FWHM) of the sample was about 70 nm. After addition of NaOH or 4-EP to the sample, UV-vis absorption spectra were obtained within several minutes. The pH of the gold nanoparticle solution was calibrated using an Orion 2-star Benchtop® pH meter (available from Thermo Electron Corp.).

The measurements of light scattering and zeta-potential were carried out using a particle size analyzer (FDLS-3000® available from Otsuka Electronics Co., Ltd.). Raman spectra were obtained using a Renishaw Raman confocal system 1000 spectroscope equipped with an integrated microscope (Leica DMLM). Spontaneous Raman scattering was obtained from a Peltier-cooled (−70° C.) charge-coupled device (CCD). The holographic super notch filter suitable for the spectroscopy was set at 632.8 nm. The spectral resolution of hologram grating (1800 grove/nm) and slit was 1 cm⁻¹. The 632.8 nm radiation from an air-cooled He—Ne laser (35 mW, Melles Griot Model 25 LHP 928) coupled with a plasma line rejection filter was used as an excitation source for the Raman experiments. Data acquisition time used in the Raman Laser irradiation was about 30 second. The Raman band of a silicon wafer at 520 cm⁻¹ was used to calibrate the spectrometer.

Colorimetric pH Indicator

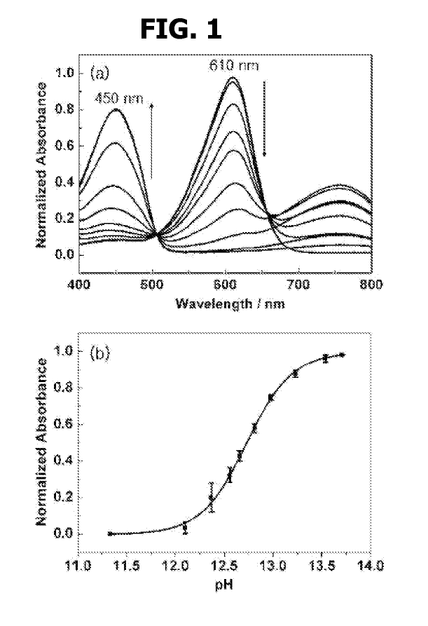

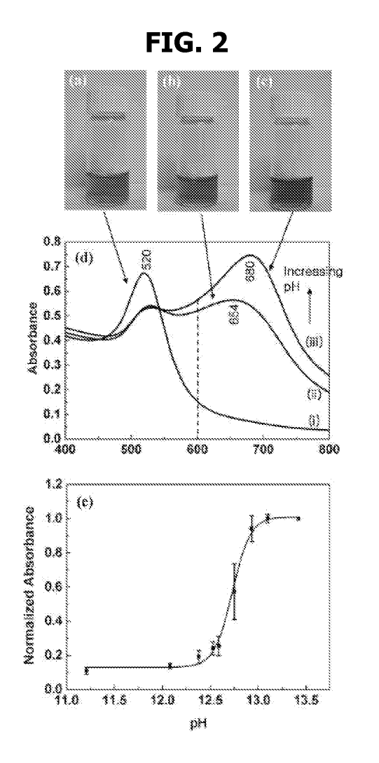

Colorimetric indicators e.g. thymolphthalein (pH 9.4-10.6), alizarin yellow (pH 10.1-12.0) and indigo carmine (pH 11.4-13.0) were used to check pH variation in alkaline regions⁽³¹⁾. The visible absorption spectra and pH titration curve obtained at 610 nm using indigo carmine that is a general calorimetric indicator are shown in FIGS. 2A and 2B, respectively. It can be seen from FIG. 1A that the absorption band at around 610 nm is sharply decreased in a highly alkaline region of pH 12-14, as the pH value increases. The pH calibration curve obtained from the band intensities was plotted in FIG. 1B. The curve shows a sharp increase of absorbance in a highly alkaline region (pH >12.2).

Surface Plasmon Resonance (SPR) Spectrum of Gold Nanoparticles

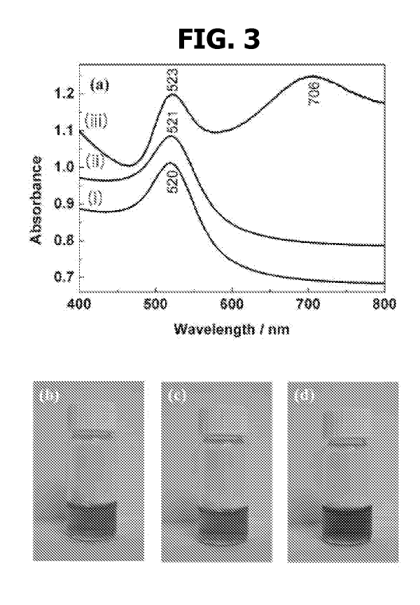

To confirm usefulness of gold nanoparticles as a pH sensor, UV-vis absorption spectra were monitored in accordance with pH variation. The results are shown in FIGS. 3A to 3C. As shown in FIGS. 3A to 3C, the gold nanoparticle solution exhibited a distinct color change upon addition of NaOH. In a region of pH <11, no precipitation was observed and the sol was red. This phenomenon was consistent with the previous reports^{(4,16)}. However, at pH about 12.5 and about 13.5, the sol exhibited purple and greenish black, respectively. These results indicated that replacement of trivalent citrate ions adsorbed on the gold nanoparticle surfaces by monovalent hydroxide ions makes the nanoparticles unstable, causes nanoparticle aggregates, and thus leads to an increase in the size of the nanoparticles. In addition, the measurement of light scattering and zeta potential demonstrated that addition of the hydroxide ions to the sol medium caused aggregation of the gold nanoparticles and variation in surface potential of the gold nanoparticles.

As shown in FIG. 2(d), aggregation of the gold nanoparticles during a self-assembly process can be confirmed from red shift in UV-vis absorption spectra that results from a reduction in the distance between nanoparticles. These results appear to be extended from those reported by the inventors. After the addition of NaOH, red shift of the surface plasmon bands indicates that larger aggregates were created. Aggregation of gold nanoparticles can be seen from characteristic behaviors of UV-vis absorption spectra at a wavelength of 600 nm or more. The extent of the aggregation was evaluated by calculating the integrated values of absorbance in a wavelength range from 600 to 800 nm. As a result, the aggregation extent increases as a function of time, and reaches a maximum value at a specific point or above. The titration according to pH variation based on the integrated value of absorption spectra is plotted in FIG. 2(e). Taking into consideration the fact that the sol solution of gold nanoparticles was prepared from 1.28×10⁻³ M KAuCl₄, the ionic strength of the gold nanoparticles was 2.0×10⁻² M. As shown in FIG. 2, according to the invention, citrate-induced gold nanoparticles have different absorbance in three regions of pH <11, 11<pH<13 and pH >13. In particular, it can be seen from the titration curve shown in FIG. 2(e) that the absorbance sharply increases at pH 12.5 to 13.0. Accordingly, this means that gold nanoparticles reduced by citrate can be used as a pH sensor via color change or absorbance observation.

Evaluation for Effects of 4-ethynylpyridine Self-Assembly Monolayer on Gold Nanoparticles

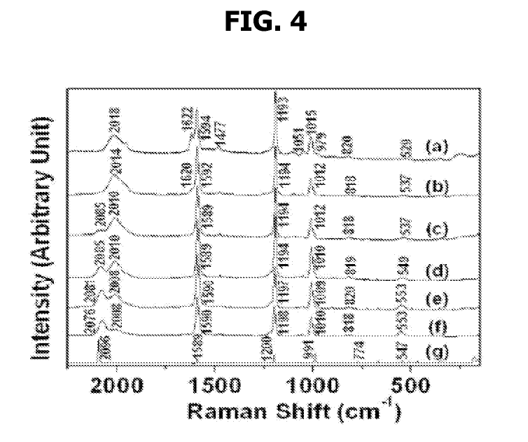

4-ethynylpyridine (4-EP) is bound to the surface of a metal in several manners by means of a pyridine ring or acetylene group. FIG. 3(a) shows surface plasmon resonance spectra before and after addition of NaOH. FIGS. 4B to 4D show color variations of the gold nanoparticle solution, before and after addition of NaOH. It can be seen from UV-vis data that plasmon red shift is caused by hydroxyl (OH—) ions, rather than 4-EP. Even in the absence of 4-EP, gold nanoparticles are aggregated in highly alkaline regions, as shown in FIG. 2.

Raman Spectra of 4-ethynylpyridine (4-EP)

To investigate adsorption behaviors of 4-EP in more detail, SERS spectra were obtained in various pH regions. FIG. 4 shows Raman spectra of 4-EP in a liquid phase and SERS spectra of gold in various pH regions. The surface area and volume of colloidal gold nanoparticles were estimated to be 707 nm² and 1,770 nm³, respectively, from the average diameter (about 15 nm) thereof. Taking into consideration the fact that the diameter of gold atoms is 0.14425, the number of gold atoms in each nanoparticle is 1.41×10⁵. Taking into account the fact that each 4-EP molecule takes up an area of 0.217 nm² in a perpendicular arrangement, 3,260 4-EP molecules are required to cover each gold nanoparticle. The 4-EP concentration in the gold nanoparticle solution required to cover colloidal gold nanoparticles is 3.0×10⁻⁵ M, i.e. 3,260×9.1×10⁻⁹ M⁽³⁰⁾. The concentration of 4-EP in the gold nanoparticle solution is about 10⁻⁴M, thus being sufficient to form a self-assembly monolayer.

To acquire information associated with surface mechanism, it is necessary to analyze spectral variation in the process of adsorption. Raman spectra of FIG. 4 were analyzed on the basis of vibrational assignment^{(14,25)}. It is considerably simple to correlate ordinary Raman (OR) bands with Au SERS bands. The peak positions and vibrational assignments are given in Table 1.

TABLE 1

| | | |
|---|---|---|
| | Au SERS | |
| Ordinary Raman | (~10⁻⁴ M, at pH ~6.7) | Assignment |

In-Plane

| | | |
|---|---|---|
| 1589 | 1589 | 8a (A₁) |
| | 1479 | 19a (A₁) |
| 1200 | 1194 | 9a (A₁) |
| | 1120 | 18a (A₁) |
| | 1010 | 12 (A₁) |
| | 1006 | 18b (B₂) |
| 991 | 995 | 1 (A₁) |
| 670 | 665 | 6b (B₂) |
| 466 | | 6a (A₁) |

Out-of-Plane

| | | |
|---|---|---|
| 774 | 819 | 11 (B₁) |
| 509 | 488w | 16b (B₁) |

Anchoring Group

| | | |
|---|---|---|
| 2096 | 2085 | |
| | 2010 | v(C≡C) |
| 547 | 549w | β(C—C≡C) |
| 469 | | α(C—C—C) |
| | 344 | β and γ(C—CCH) |

FWHMs of free ν(C≡C) bands became broad upon adsorption of 4-EP on gold nanoparticles. This indicates that 4-EP is bound to gold surfaces by means of acetylene. As shown in FIG. 4, strong intensities of in-plane ring modes mean that 4-EP assumes a perpendicular orientation with respect to the gold surface.

On the assumption that long-range electromagnetic (EM) effects and short-range chemical effects exist at the same time, qualitative analysis only associated with information obtained from SERS has been mentioned⁽³²⁻³⁴⁾. On the basis of electromagnetic surface selection rules⁽³²⁻³⁴⁾, a simple interfacial structure of an aromatic adsorbate on gold and silver surfaces was quantitatively explained⁽³⁴⁾. An additional contribution to the SERS phenomenon is a charge transfer (CT) mechanism considered as analogous to a resonance Raman process, although it strongly depends on the nature of the metal-adsorbate system without general rules⁽³²⁾. The simple analysis for the 4-EP structure can be conducted at several selected peaks, based on estimations from the electromagnetic surface selection rules. According to EM selection rules, vibrational modes perpendicular to the surface show stronger SERS intensities, as compare to those parallel to the surface. In a case of 4-EP, the most intensified ring modes belong to in-plane modes except for specific patterns at about 819 cm⁻¹ and about 488 cm⁻¹. The weakening of out-of-plane band intensities indicates that the adsorbate assumes a perpendicular orientation with respect to the metal surface. The CT mechanism was reported to substantially contribute to an increase in SERS sensitivity⁽³³⁾. The characteristic patterns of SERS spectra can be schematically illustrated by EM mechanism. Further, CT mechanism also contributes to SERS intensities in several vibrational bands of 4-EP on gold surfaces. It was evaluated that the most intensified vibrational mode by CT mechanism is V₈ₐ. It's very interesting that V₈ₐ was greatly intensified at SERS spectra of 4-EP on gold surfaces. To monitor adsortive behaviors of acetylene on gold surfaces, more research is demanded. Taking into consideration the fact that spectral characteristic patterns significantly vary depending on pH in v(C≡C) stretching regions of SERS spectra with respect to the gold nanoparticle surfaces, multiple bands can be ascribed to in-plane adsorption of several complexes or other crystals. On the other hand, it is worthy of notice that in-plane ring modes showed no change in very low pH (<0.8) ranges except for two peaks of 1,620 and 1,051 cm⁻¹. This indicates that the perpendicular orientation of the pyridine ring is maintained in several binding patterns with acetylene.

As shown in FIG. 4, vibrational modes of 4-EP are greatly affected by pH variation. Ionization of pyridine and acetylene in 4-EP is varied depending on pH conditions. According to previous reference documents^{(14, 15)} associated with 4-mercaptopyridine, the stretching mode of the pyridine ring is observed at 1,620 cm⁻¹ under acidic conditions (pH <6) due to hydrogenation of the pyridine ring. In addition, this spectral variation was observed under acidic conditions at SERS spectra of 4-EP. It is notable that v(C≡C) stretching peaks are shifted as a function of pH, in particular, under alkaline conditions.

ν(C≡C) Stretching Band

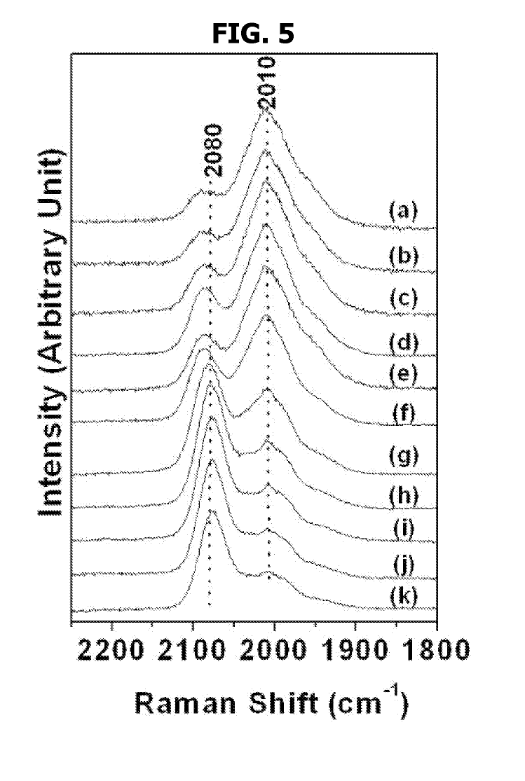

ν(C≡C) stretching peaks on gold nanoparticle surfaces show a multi-structure upon adsorption. As shown in FIG. 4, ν(C≡C) stretching bands vary significantly in highly alkaline regions of pH >11, in particular, pH 11.5 to 14. FIG. 5 shows a enlarged view of a SERS spectral variation of ν(C≡C) stretching bands according to pH variation. According to previous reports by the inventors, when NaBH₄, KCl and KBr are added to a sol medium, ν(C≡C) stretching bands on gold nanoparticle surfaces vary significantly. The addition of BH₄ causes a decrease in band intensities from about 2,015 cm⁻¹ to about 1,960 cm⁻¹. On the other hand, in a case where the sol medium becomes an alkaline solution, ν(C≡C) stretching bands of 4-EP are gradually strengthened at 2,080 cm⁻¹ and gradually weaken at 2,010 cm⁻¹. These results indicate that the pH value of the sol medium greatly affects formation of self-assembled monolayers (SAMs) composed of an aromatic compound (i.e. a pyridine compound) which is bound via acetylene as an anchoring group on gold nanoparticle surfaces. As the pH value increased, the v(C≡C) stretching band intensity at ˜2,010 cm⁻¹ was gradually decreased in alkaline regions as well as acidic regions. As the pH value increased, the v(C≡C) stretching band intensity at about 2,080 cm⁻¹ was gradually increased. In conclusion, characteristic peaks of the v(C≡C) stretching bands vary depending on the pH value, and SAMs of 4-EP on Au nanoparticles held potential as a pH sensor in highly alkaline regions.

Surface Enhancement Raman Scattering (SERS) Titration of 4-ethynylpyridine as pH Sensor

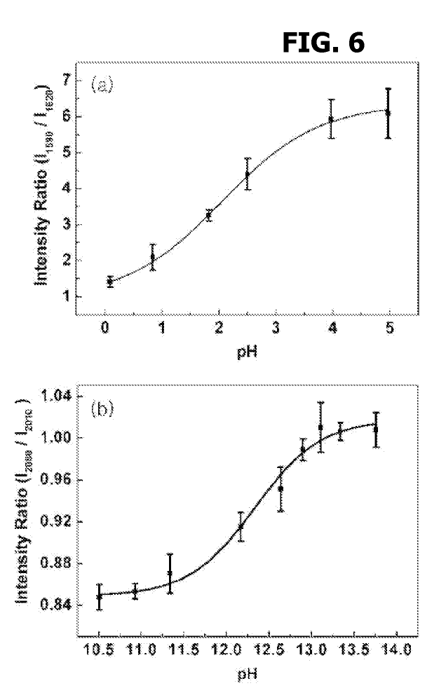

S-shape pH titration curves shown in FIGS. 6(a) and 6(b) were obtained from measurements of peak intensity ratios of I₁₅₉₀/I₁₆₂₀ and I₂₀₈₀/I₂₀₁₀. The pH calibration from SERS titration of 4-EP has advantages in terms of higher alkaline detection limit and more precise measurements, as compared to indigo carmine shown in FIG. 1. From the calibration curve, the pK_{1/2} value was determined to be around 13.

According to Gouy-Champman-Stem theory^{(12,13)}, local pH_{bulk} and pH_{surface} values were given from Equation 1 below:

pH_{surface}=pH_{bulk}+eψ/2.3 kT (1)

In Equation 1, e: electric charge, k: Boltzmann constant, T: temperature, and ψ: surface potential.

It can be confirmed from zeta potential measurement⁽⁸⁾ that the surface potential is about −50 mV and pH_{surface} is about 14. The pKₐ value of acetylene contained in neutral phenyl acetylene is about 30. There is no clear reason why the high pKₐ value is significantly decreased on gold nanoparticle surfaces. The aforementioned result means that gold nanoparticles can be employed as a pH indicator, and furthermore, they can be employed as a pH indicator or in pH calibration in highly alkaline regions of pH >11, preferably, pH 11.5 to 14, most preferably, pH 12 to 14.

As apparent from the foregoing, it was confirmed that gold nanoparticles can be used as a pH indictor in highly alkaline regions (i.e. pH >11) by means of SERS spectroscopic instruments. Citrate-reduced gold nanoparticles exhibit a distinct color change in a highly alkaline region. As the pH value increased, the v(C≡C) stretching band intensity at about 2,010 cm⁻¹ was gradually decreased in highly alkaline regions, but the v(C≡C) stretching band intensity at about 2,080 cm⁻¹ was gradually increased under the same conditions. In conclusion, SERS titration via the v(C≡C) stretching bands enables pH calibration in highly alkaline regions as well as more precise pH measurement, as compared to conventional indicators.

Although the preferred embodiments of Example embodiments have been disclosed for illustrative purposes, those skilled in the art will appreciate that various modifications, additions and substitutions are possible, without departing from the scope and spirit of the invention as disclosed in the accompanying claims.

### REFERENCES

1. A. N. Shipway, E. Katz, and I. Willner, ChemPhysChem 1, 18 (2000).
2. U. Kreibig and M. Volmer, Optical Properties of Metal Clusters, (Springer, Berlin, 1995).
3. F. E. Osterloh, H. Hiramatsu, and R. Porter, T. Guo, Langmuir 20, 5553 (2004).
4. C. S. Weisbecker, M. V. Merritt, and G. M. Whitesides, Langmuir 12, 3763 (1996).
5. H. Fleischmann, P. J. Weaver, and A. J. McQuillan, Chem. Phys. Lett. 26, 163 (1974).
6. X. Su, J. Zhang, L. Sun, T-W. Koo, S. Chan, N. Sundararajan, M. Yamakawa, and A. A. Berlin, Nano Lett. 5, 49 (2005).
7. R. Stosch, A. Henrion, D. Schiel, and B. Güttler, Anal. Chem. 77, 7386 (2005).
8. T. Kim, K. Lee, M-s. Gong, and S-W. Joo, Langmuir 21, 9524 (2005).
9. M. A. Bryant and R. M. Crooks, Langmuir 9, 385 (1993).
10. X. Zhao, S. Ong, H. Wang, and K. B. Eisenthal, Chem. Phys. Lett. 214, 203 (1993).
11. D. V. Vezenov, A. Noy, L. F. ozsnyai, and C. M. Lieber, J. Am. Chem. Soc. 119, 2006 (1997).
12. K. I. Mullen, D. Wang, G. Crane, and K. T. Carron, Anal. Chem. 64, 930 (1992).
13. H-Z. Yu, N. Xia, and Z-F. Liu, Anal. Chem. 71, 1354 (1999).
14. H. S. Jung, K. Kim, and M. S. Kim, J. Mol. Struct. 407, 139 (1997).
15. J. Hu, B. Zhao, W. Xu, B. Li, and Y. Fan, Spectrochim. Acta A 58, 2827 (2002).
16. R. A. AlvarezPuebla, E. Arceo, P. J. G. Goulet, J. J. Garrido, and F. Aroca, J. Phys. Chem. B 109, 3787 (2005).
17. J. C. Love, L. A. Estroff, J. K. Kriebel, R. G. Nuzzo, and G. M. Whitesides, Chem. Rev. 105, 1103 (2005).
18. R. Dagastine and F. Grieser, Langmuir 20, 6742 (2004).
19. S. W. Han, S-W. Joo, T. W Ha, Y. S. Kim, and K. Kim, J. Phys. Chem. B 104, 11987 (2000).
20. N. J. Long and C. K. Williams, Angew. Chem. 42, 2586 (2003).
21. M. J. Ford, R. C. Hoft, and A. McDonagh, J. Phys. Chem. B 109, 20387 (2005).
22. F. F. Fan, J. Yang, L. Cai, D. W. Price, M. D. Shawn, D. V. Kosynkin, Y. Yao, A. M. Rawlett, J. M. Tour, and A. J. Bard. J. Am. Chem. Soc. 124, 5550 (2002).
23. H. Feilchenfeld and M. J. Weaver, J. Phys. Chem. 93, 4276 (1989).
24. L. M. Abrantes, M. Fleishmann, I. R. Hill, L. M. Peter, M. Mengoli, and G. Zotti, J. Electroanal. Chem. 164, 177 (1984).
25. S. W. Joo and K. Kim, J. Raman Spectrosc. 35, 549 (2004).
26. Y. T. He and S. J. Traina, Environ. Sci. Technol. 39, 4499 (2005).
27. B. Borucki, H. Otto, and M. P. Heyn, J. Phys. Chem. B 108, 2076 (2004).
28. P. C. Lee and D. Meisel. J. Phys. Chem. 86, 3391 (1982).
29. S. W. Joo, S. W. Han, and K. Kim, J. Phys. Chem. B 103, 10837 (1999).
30. S-W. Joo, W-J. Kim, W. S. Yoon, and I. S. Choi, J. Raman Spectrosc. 34, 271 (2003).
31. D. C. Harris, Quantitative Chemical Analysis, (W. H. Freeman and Company, New York, 1982).
32. M. Moskovits, Rev. Mod. Phys. 57, 783 (1985).
33. G. C. Schatz and R. P. Van Duyne, in Handbook of Vibrational Spectroscopy edited by J. M. Chalmers and P. R. Griffiths, (John Wiley & Sons, New York, vol. 1, pp 759-774, 2002).
34. S-W. Joo, Vib. Spectrosc. 34, 269 (2004).

What is claimed is:

1. A pH sensor for use in a highly alkaline region of pH >11 comprising citrate-reduced gold nanoparticles.

2. The pH sensor according to claim 1, wherein the gold nanoparticles have a surface on which a self-assembled monolayer composed of a compound having a pyridine ring and an acetylene group as an anchoring group is formed.

3. The pH sensor according to claim 1, wherein the compound is 4-ethynylpyridine.

4. A method for calibrating pH of a solution in a highly alkaline region of pH >11, the method comprising the steps of:
adding citrate-reduced gold nanoparticles to a target sample;
obtaining surface-enhanced Raman scattering spectra from the sample; and
calibrating pH of the sample via measurement of v(C≡C) stretching band intensity from the spectra,
wherein each citrate-reduced gold nanoparticle has a surface on which a self-assembled monolayer composed of a compound having a pyridine ring and an acetylene group as an anchoring group is formed.

5. The method according to claim 4, wherein the compound is 4-ethynylpyridine and the highly alkaline region ranges from pH 11.5 to 14.

6. The method according to claim 4, wherein the measurement of v(C≡C) stretching band intensity is carried out by calculating an intensity ratio between two characteristic peaks.
